(19) United States
(12) Patent Application Publication
(10) Pub. No.: US 2002/0025537 A1
(43) Pub. Date: Feb. 28, 2002

(54) High-throughput methods for generating and screening compounds that affect cell viability

(76) Inventors: Edward J. Bylina, San Diego, CA (US); William J. Coleman, San Diego, CA (US); Douglas C. Youvan, San Diego, CA (US)

Correspondence Address: Michael J. Shuster, Ph.D., McCutchen, Doyle, Brown & Enersen, LLP, 28th Floor, Three Embarcadero Center, San Francisco, CA, 94111, US

(21) Appl. No.: 09/909,652

(22) Filed: Jul. 19, 2001

Related U.S. Application Data
(60) Provisional application No. 60/219,179, filed on Jul. 19, 2000.

(51) Int. Cl. C12Q 1/68; G01N 33/53; C12Q 1/02

(52) U.S. Cl. 435/7.1; 435/29; 435/6

(57) ABSTRACT

The invention relates generally to the area of drug development and more specifically to screening novel compounds for antimicrobial activity. Methods are described for generating mutagenized peptide libraries expressed in colonies of cells. A fluorescent live/dead cell assay combined with a digital imaging spectrophotometer provides a high-throughput solid-phase screening method for colonies or arrayed synthetic libraries. The assay enables screening of antimicrobial peptide activity in at least 10⁵-10⁶ colonies per experiment. These methods generate and identify antimicrobial compounds.

### CROSS-REFERENCE TO RELATED APPLICATIONS

This applications claims the benefit of U.S. Provisional Application No. 60/219,179, filed Jul. 19, 2000 the entire disclosure of which is herein incorporated by reference in its entirety.

### STATEMENT REGARDING FEDERALLY SPONSORED RESEARCH

Not applicable.

### FIELD OF THE INVENTION

The invention relates to methods for high-throughput screening and drug discovery.

### BACKGROUND OF THE INVENTION

The use of antibiotics in medical practice and agriculture has led to the proliferation of multi-drug resistant microbial strains. As many as 90% of isolates from hospital-acquired bacterial infections display resistance to one or more conventional antibiotics (Kelley et al., 1996). Even resistance to vancomycin—one of the antibiotics of last resort for Gram positive bacteria—is spreading. Recently, strains of Staphylococcus aureus with reduced susceptibility to vancomycin have been identified (Hiramatsu, 1998; Domin, 1998), and the appearance of vancomycin resistant strains of enterococci has given rise to some infections that are virtually untreatable. As more and more pathogens become resistant to currently available antibiotics, there is a need to develop new classes of antibiotics, such as cationic antimicrobial peptides. But in order to find new classes of antimicrobial peptides, there is a need in the art for new methods to identify such compounds. Screening libraries of peptide variants for antimicrobial activity is a promising avenue for exploration; however, the sequence space that can be searched is enormous.

Comparison with Other Peptide Library Methods

The work of Mekalanos and Blum (Blum et al., 2000 and WO 99/50462) describes methods for creating libraries of peptide “aptamers” for discovering antimicrobial compounds. Their methods fail to address some of the deficiencies of the prior art addressed by the present invention. For example: (1) The aptamers are defined to be relatively short peptides (16 residues in the example given, but anywhere from 7 to 80 residues theoretically) that are expressed as fully constrained loops. (2) The libraries of aptamers used by Mekalanos and Blum, (which are defined as “random combinatorial peptide sequences”) are generated by using fully random combinatorial cassette mutagenesis. (3) In the methods of Mekalanos and Blum the ‘positive’ aptamers are identified by their killing or inhibiting the host in which they are expressed. (4) Mekalanos and Blum employ a plus/minus scheme requiring laborious growth selection replica plating to identify ‘positive’ aptamers.

The work of Taguchi et al. (1994;1996) describes methods for determining the functional amino acids in the 18-residue antimicrobial peptide apidaecin. These authors fused the nucleotide sequence for apidaecin to the gene sequence for the Streptomyces subtilisin inhibitor (SSI) protein contained in a plasmid vector. The majority of the apidaecin-SSI insert was then subjected to error-prone PCR mutagenesis to create a mutagenized library. After transforming an E. coli host, the transformants were plated and the colonies were visually screened for growth inhibition. Cells expressing inhibitory peptides were also identified by liquid growth assays. Thus, the method of Taguchi et al. also fails to address certain deficiencies of the art in that: (1) The peptides are not cleavable from the carrier protein. (2) The library of mutagenized peptides was created by error-prone mutagenesis of both the carrier protein and the peptide. Mutations are therefore not necessarily localized to the peptide alone. (3) The mutagenesis is not targeted to any particular region of sequence space, but instead relies on random misincorporation of nucleotides. (4) The assay depends on limited growth inhibition of the host cells in which the peptides are expressed. Thus, it is not possible to identify peptides which killed the host cell.

Efficient methods are therefore needed to create libraries having optimal (not just maximal) diversity. Likewise, it is important to have methods for activity screening which can operate at high throughput and identify peptide variants possessing a desired activity. The methods described in the present invention are designed to achieve these goals.

### SUMMARY OF THE INVENTION

The present invention describes methods for generating complex libraries of peptides expressed in colonies or microcolonies and methods for high-throughput screening to identify antimicrobial compounds. Recursive Ensemble Mutagenesis (REM)—a mutagenesis strategy that uses a biological embodiment of a genetic algorithm—is used to generate a highly complex library of peptide variants. Expression of the peptide library can be enhanced by fusing the peptide to a carrier protein. Fusion allows high-level expression of the peptide in a more soluble form and reduces the level of degradation of the peptide in the host organism. Examples of carrier proteins include ubiquitin, maltose binding protein, cell surface display vehicles, bacteriophage coat proteins, and the like. An inducible expression system using antimicrobial peptides fused to ubiquitin makes it possible to controllably express active antimicrobial peptides in E. coli or other expression systems. The peptide can subsequently be released from the carrier by treatment with a sequence-specific endopeptidase. This technique makes it possible to assay the authentic peptide sequences contained in the library for antimicrobial activity by means of a viability screen.

The present invention also provides methods for distinguishing dead cells (expressing active sequences) from living cells (expressing inactive or less active sequences) by means of colorimetric solid-phase screening. The high-throughput screening assays comprise the use of various dyes to monitor the viability of the target cells. Viability of the target cells can be monitored using a digital imaging spectrophotometer known as the MicroColonyImager. This method can be used to screen greater than about 10⁵ to 10⁶ microcolonies per experiment. Members of the library that appear to be candidate positives can be retrieved and sequenced. The combined mutagenesis and screening strategy makes it possible to identify novel antimicrobial peptide sequences, including highly potent molecules, resulting in a large number of new antimicrobial lead compounds that are active against a broad range of bacteria or other microorganisms. The solid-phase assay method is generally useful for screening all types of antibiotic compounds, including libraries of low molecular weight molecules produced by metabolic engineering and artificially synthesized libraries in solid-phase arrays.

In one embodiment of the present invention, the peptides are expressed as carboxy-terminal fusions. In another embodiment, the peptides may be expressed as amino-terminal fusions. Attachment of the foreign peptide to the terminus of the carrier protein means that the peptide sequence is not internal to the carrier protein sequence. Thus in an embodiment of the invention, the peptides used in the methods of the present invention (a) are unconstrained, (b) can have almost any length, (c) can be fused to a variety of carrier proteins, and (d) do not require the carrier protein for activity (i.e., they can be cleaved off the C-terminus). In an embodiment of the present invention, targeted (recursive ensemble) mutagenesis is used to generated an expression library. Because REM can be used iteratively, it means that successive rounds of mutagenesis and screening can be used to target (and progressively narrow down) regions of sequence space encoding potentially active peptides. In an embodiment of present invention, the candidate antimicrobial peptides may be expressed in a host organism but screened for activity against a second target organism by means of a ‘sandwich’ assay. In an embodiment of the invention an optical assay is used that describes compounds for finding compounds which employs fluorescent or other viability indicators and imaging and avoids growth selection schemes or replica plating. This provides a rapid, flexible, high-throughput system for finding candidate antimicrobial peptides.

### BRIEF DESCRIPTION OF THE DRAWINGS

The file of this patent contains at least one drawing executed in color. Copies of this patent with the color drawings will be provided by the Patent and Trademark Office upon request and payment of the necessary fee.

FIG. 1 Flow chart for Recursive Ensemble Mutagenesis (REM), a technique based on the iterative use of combinatorial mutagenesis. This technique can be used for directed evolution of peptides.

FIG. 2 Differences in fluorescence emission of E. coli suspensions containing various percentages of live and dead cells after simultaneous staining with SYTO 9 and propidium iodide. Live cells emit green fluorescence (G) in the presence of SYTO 9, and dead cells emit red fluorescence (R) in the presence of propidium iodide (Haugland, 1996).

FIG. 3 Graphical User Interface (GUI) of the MCI instrument in the absorption mode. Approximately 10,000 colonies have been generated on the surface of a disk (upper left window). A few ‘positive’ microcolonies displaying unusual spectra have been sorted and identified in a ‘color contour plot’ (right window) where the spectrum of each pixel is displayed as a row. Approximately 10,000 single-pixel spectra are concisely displayed, each mapped to a microcolony. The absorbance at a given wavelength for each pixel is represented by a blue/black (low) to pink/white (high) color code. The spectra of the five circled microcolonies are displayed in the lower left plot window. Each microcolony is approximately 100 microns in diameter.

FIG. 4 Complete bovine lactoferricin sequence (bold) and amino acid variations (displayed to the right for each position) that are found in lactoferricin and magainin peptides from other species. Some sequence positions are more conserved through evolution than others. For example, Gly-14 is conserved in all of the sequences, whereas the requirement for a particular amino acid at position 8 appears to be less stringent. This consensus sequence can be used to determine the training set of amino acids in the first round of REM. Sequences of the active peptides obtained in the first round of REM can be combined into the known sequences to create a more complete training set of amino acids for further REM iterations.

FIG. 5 Optimized nucleotide mixtures for the 25 sequence positions shown in FIG. 4. The number to the right of each triplet mixture indicates the complexity per codon. The product of these 25 individual values is the overall maximum complexity of the library.

FIG. 6 Cloning region at the end of ubiquitin. Sac II-Bgl II cassettes can be used for inserting antimicrobial peptide sequences at the C-terminus of ubiquitin. The specific cassette shown here includes the DNA sequence encoding indolicidin. The peptide sequences of CEMA and lactoferricin are also shown.

FIG. 7 Overview of the antimicrobial peptide cloning and screening strategy. The procedure converts a (doped) oligonucleotide into duplex DNA that can be efficiently ligated into a plasmid and expressed at high levels in an E. coli background. Screening is performed using the MicroColonyImager.

FIG. 8 Demonstration of the SYTOX Green colony viability assay. Microcolonies containing mutagenized beta-lactamase genes are exposed to ampicillin and then assayed in the presence of SYTOX Green. Panel A shows the fluorescence image of the filter after 30 minute incubation with SYTOX Green. Cells in fluorescent microcolonies have been lysed by exposure to the antibiotic. Panel B shows all the microcolonies on the filter using 610 nm scattering. Panel C shows the microcolonies expressing beta-lactamase by following the absorption of nitrocefin hydrolysis at 550 nm. Panel D is a pseudocolored image combining Panels A and C. The results shown in Panel D confirm that fluorescent microcolonies (dead) did not catalyze nitrocefin hydrolysis, and that microcolonies which catalyzed nitrocefin hydrolysis (alive) were not stained with SYTOX Green.

### DETAILED DESCRIPTION OF THE INVENTION

The present invention provides a method for generating highly complex libraries of peptides that can be screened for antimicrobial activity. Generating a peptide library comprises the step of fusing the peptide sequences to a carrier protein such as ubiquitin so that the library can be efficiently expressed in E. coli. It further provides high-throughput solid-phase methods for screening these libraries to find peptides that possess high activity. These peptides are useful as antimicrobial agents in the pharmaceutical industry, and will provide an additional new class of antibiotic compounds to fight infectious diseases. The high-throughput screening method is also generally applicable to assaying libraries of compounds for antimicrobial activity.

Unless otherwise defined, all technical and scientific terms used herein have the same meaning as commonly understood by one of ordinary skill in the art to which this invention belongs. Methods and materials similar or equivalent to those described herein can be used in the practice or testing of the present invention, which is illustrated by the description of suitable methods and material below.

All publications, patent applications, patents, and other references mentioned herein are incorporated by reference in their entirety. In the case of conflict, the present application, including definitions, will control. In addition, the materials, methods, and examples described herein are illustrative only and are not intended to be limiting.

Other features and advantages of the invention will be apparent from the following detailed description, the drawings, and from the claims.

Antimicrobial Peptides

Antimicrobial peptides are produced by most organisms, including humans, and are a component of their natural defense against microbes (Hoffman et al., 1999; Hancock, 1997a; Boman, 1995). The majority of peptides studied to date have membrane-disruptive properties which appear to contribute to their lethality. These peptides form holes in the cell membranes of susceptible microbial cells. This permeabilizes the membrane and leads to rapid cell lysis and death. Antimicrobial peptides exhibit varying degrees of antibacterial, fungicidal, antiviral, and tumoricidal activities, making them attractive candidates for drug development. As used in the present invention, “antimicrobial” is intended to include compounds that affect the viability of bacterial, viral, fungal, or tumor cells.

Research reports and the results of various clinical trials support the potential for this class of antimicrobials (Hancock & Chapple, 1999). The in vitro and in vivo activity of protegrin-1 (PG-1), a native peptide obtained from porcine leukocytes, has been evaluated (Steinberg et al., 1997). Researchers were able to demonstrate rapid bactericidal activity against both Gram negative and Gram positive pathogenic bacteria, including strains of methicillin resistant S. aureus and vancomycin resistant E. faecalis. More importantly, administration of PG-1 intravenously protected mice from lethal challenges with the latter two agents, without apparent toxicity to the mice. The anti-fungal peptide Mycoprex (one of a number of ‘bactericidal/permeability-increasing protein’ (BPI)-derived bioactive peptides produced by Xoma, Berkeley, Calif.) is in advanced preclinical testing. Nisin (a lantibiotic cationic peptide produced by AMBI, Purchase, N.Y.) has undergone Phase I clinical safety trials successfully and is being considered for use in the treatment of H. pylori induced stomach ulcers. MBI 226 (a bactolysin peptide from Micrologix Biotech, Vancouver, B.C.) has successfully completed a two-part Phase I human clinical trial and is being considered for prevention of bloodstream infections in patients undergoing central venous catheterization. IB-367 (a protegrin-like cationic peptide from Intrabiotics, Mountain View, Calif.) has also passed Phase II testing for topical oral use and is currently under evaluation for clinical safety as an aerosol. IB-367 is being considered for the treatment of both oral mucositis caused by cancer chemotherapy and P. aeruginosa lung infection in patients with cystic fibrosis.

Antimicrobial peptides can be divided into four major structural classes: (1) beta-sheet structures with multiple disulfide bonds, such as the defensins; (2) alphahelical structures, such as the cecropins and magainins; (3) extended coils with a predominance of one or more amino acids, such as indolicidin, and (4) loop structures with a single disulfide bond, such as lactoferricin. Synthetic D-enantiomers of certain of these peptides maintain their function, indicating that peptide activity is not dependent on chiral interactions with the membrane (Merrifield et al., 1994). The presence of D-amino acids would make such peptides highly resistant to proteolysis, and therefore theoretically more stable in vivo. Certain peptides have been shown to enhance the activity of conventional antibiotics (Darveau et al., 1991; Vaara & Porro, 1996), presumably by increasing the permeability of the outer-membrane. This synergy with classical antibiotics may allow peptides to serve as anti-resistance compounds. Mutations which convey resistance to antimicrobial peptides in sensitive strains have not yet been observed (Hancock, 1997b). Due to the achiral mechanism of action of these peptides, generating resistance to them may require altering the structure of the cell membranes within the target microbe. The low probability of this global alteration occurring (compared to the easier route of simply mutating a single target protein) may prevent the rapid emergence of resistance.

Library Screening

One powerful method for discovering new chemical or biological compounds involves screening a library. A library consists of a collection or population of members that are physically or chemically distinguishable, such as different DNA, RNA, peptide, or other polymer sequences. Because many of these polymer molecules can be amplified, each member typically comprises a plurality of identical molecules which are unique to that member. The number of unique members contained in the library is referred to as the complexity. Screening involves assaying a large number of samples (i.e., members of a library) to identify candidate compounds having a desired activity or property. Often, the desired activity or property is indicated by an optical signal, such as a difference in absorbance or fluorescence emission among the various samples.

For example, members of a chemical library (comprising artificially synthesized molecules, such as peptides) can be screened by spotting them onto a surface to create an array, initiating a reaction by contacting them with one or more optical indicator compounds, and optically monitoring any subsequent changes in the optical signal, which indicates the presence of the desired activity or property. A desired member of the library can then be identified based on, for example, its position in the array or by sequencing the peptide comprising the member. A surface may comprise a substantially continuous base, which can be any biologically acceptable substrate, such as a Petri dish, assay disk for growing bacteria, or an array of glass or plastic beads. A substantially continuous base allows for free diffusion of liquid throughout its surface. An example of a substantially continuous base for useful for screening according to the methods of the invention is a sheet of polymeric material. Members of a DNA library can be screened by cloning the DNA fragments into an expression vector, transforming a suitable host cell, inducing expression of the gene product encoded by the DNA fragment, and initiating a reaction which indicates the presence of the desired activity or property. By “transformation” is meant any method for introducing foreign molecules, such as DNA, into a cell (e.g., a bacterial, yeast, algal, plant, or animal cell). Lipofection, DEAE-dextran-mediated transfection, microinjection, protoplast fusion, calcium phosphate precipitation, retroviral delivery, electroporation, natural transformation, and biolistic transformation are just a few of the methods known to those skilled in the art which may be used. A desired member can then be picked, and its DNA isolated. This DNA can then be analyzed to determine the sequence of the active peptide. Methods for creating libraries, as well as cloning and expressing genes, are well known to those of skill in the art, and are described in Sambrook et al., (1989), Birren et al. (1999), Ausubel et al. (2000) and in U.S. Pat. No. 5,914,245.

In some instances, the library encodes expression products that, in turn, synthesize active compounds. An example, the polyketide synthetic enzyme system, is described below. Diversity in such a library is generated by mix-and-match techniques that vary combinations of modular components involved in polyketide synthesis. Such methods are known in the art. Accordingly, we intend the term “encoding” when used in reference to an expression library to mean directly encoding (i.e., wherein a transcription or translation product is a candidate compound) as well as to mean indirectly encoding, such as when a candidate compound is produced by a transcription or translation product directly encoded. A polyketide expression library is one example of a library that indirectly encodes candidate compounds.

Structure-function Studies of Antimicrobial Peptides

Chemical synthesis of antimicrobial peptide analogs has been used to modify the properties of these compounds, to understand their mode of action, and to generate products with greater activity. Most of these structure-activity studies have used rational design to study the effect of size, charge, hydrophobicity, and amphiphilicity on the antimicrobial activity of different peptides. These include studies of magainins (Maloy & Kari, 1995), lactoferricin (Kang et al., 1996), histatins (Helmerhorst et al., 1997) and indolicidin (Falla & Hancock, 1997). However, the amount of ‘sequence space’ searched in these traditional studies is incredibly small; most reports involve the screening of fewer than 20 peptide sequences. Novel active sequences have been identified by screening conformationally defined synthetic combinatorial libraries based on a known antimicrobial peptide (Blondelle et al., 1996). While these libraries allow the screening of thousands of peptide analogues, this level of complexity is still small compared to the complexities of peptide libraries that could be constructed using recombinant DNA techniques (which might have between 10⁶ and 10⁸ members). The number of possible amino acid sequences that must be generated in order to fully screen a small peptide is enormous (3×10¹⁹ for a 15 amino acid peptide). Therefore, screening peptide libraries requires techniques with considerably greater throughput and efficiency to find novel peptide sequences with enhanced antimicrobial activity.

Searching Sequence Space

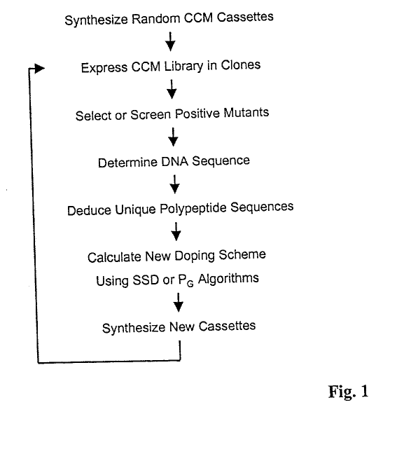

The number of possible different amino acid sequences that one can introduce into any given protein is so vast that evolving proteins with novel specificities or properties requires an efficient mutagenesis strategy in order to be practical. A variety of techniques for the generation of modified proteins (i.e., polypeptides having more than about 80 amino acids) have already been described. These include well known PCR-based methods such as DNA shuffling (Stemmer, 1994), and Sequential Random Mutagenesis, i.e., SRM (Moore & Arnold, 1996). In the case of directed evolution applied to small peptides, however, a more suitable technique is recursive ensemble mutagenesis, or REM, which is described in FIG. 1.

While REM can target entire regions of a peptide with simultaneous mutations, SRM and DNA shuffling rely on generating sets of individual point mutations one at a time. The untargeted methods thus have three major drawbacks for peptide studies: (1) The mutations are spatially random, and thus highly ‘dilute’ in three-dimensional space; (2) The mutations are generated at a relatively low frequency (˜0.5%); and (3) The polymerase is mutationally biased. Because of these limitations, it is not easy to combine several useful mutations into a short sequence, even when using recombination. This makes it difficult to isolate a functional mutant that may require several simultaneous mutations to display any gain in function. SRM and DNA shuffling also cannot take full advantage of the genetic code because single point mutants within codons generated by error-prone PCR produce only a fraction of all possible changes. In addition, error-prone PCR is not completely random (i.e., it favors mutations at A and T more than C and G; Shafikhani et al., 1997). Therefore, REM enables a more thorough search of sequence space than these other methods, and this search can be reasonably performed due to the small size of the peptides in question.

REM was first applied to the study of photosynthetic Light Harvesting (LH) peptides, whose chromophores act as convenient reporter groups for indicating changes in structure. This technique involves the recursive use of combinatorial cassette mutagenesis (CCM; Oliphant et al., 1986; Reidhaar-Olson & Sauer, 1988) to generate a diverse library of genetically altered peptides (FIG. 1). REM's ability to converge on sets of peptides with novel attributes has been demonstrated using chromogenic LHII peptides from the bacterium Rhodobacter capsulatus. ‘Positive’ clones were picked based on the in situ absorption or fluorescence properties of individual colonies measured directly on Petri dishes (Goldman & Youvan, 1992; Youvan et al., 1992; Delagrave et al., 1993; Youvan, 1994; Goldman et al., 1994; Youvan, 1995; Delagrave et al., 1995).

It is important to realize that, even with throughputs of one million colonies per day, it would still be impossible to fully randomize and screen a 25-residue peptide. The complexity of a fully randomized 25-residue peptide is 20²⁵=3.4×10³². It would take longer than the age of the universe to screen such a fully randomized library. Strategies such as REM are thus highly advantageous for effecting rapid convergence to active sequences.

REM is employed as a combinatorial mutagenesis technique in order to generate new classes of antimicrobial peptides, even when starting with a set of known peptide sequences. This approach is analogous to what has been used in other iterative mutagenesis experiments using known protein sequences. A number of DNA shuffling experiments that started with a single enzyme or a group of phylogenetically related enzymes have produced novel activities and significant changes in substrate specificity in the resulting enzymes (e.g., Joo et al., 1999). The use of REM to identify antimicrobial peptides with novel activities is limited only by the assays developed to screen for those activities.

After one walks down a protein sequence and independently optimizes different sections of the protein with REM, the technique of Exponential Ensemble Mutagenesis (EEM) can then be applied to combine the results of these separate experiments (Delagrave & Youvan, 1993). The EEM technique ultimately restricts sequence complexities over longer sequences and provides a method for the variable sites to be re-optimized by constructing a combinatorial cassette to cover the entire sequence region of interest. Mutagenesis can be carried out on either contiguous segments of the peptide or on various residues interspersed throughout its sequence. Based on the LHII experimental results, this method yields a 10¹³-fold ‘gain’ over fully random CCM for a 30 residue peptide. Practically, this means that randomizing a 30-mer with CCM is likely to yield no positive clones, while the use of iterative techniques such as REM and EEM yields a library of functionally distinct and diverse peptides.

Expression of Antimicrobial Peptide Libraries

Up to now, the power of recombinant DNA procedures has not been effectively exploited for producing antimicrobial peptides in bacteria. The expression of antimicrobial peptides is problematic because the desired product is often toxic to the host, such as E. coli. Fusion proteins containing a desired peptide sequence lose their antimicrobial activity (Piers et al., 1993), but the positively charged peptides—even when part of certain fusion proteins—are often sensitive to proteolysis by endogenous proteases in the host. A system for peptide production has been developed wherein an engineered fusion protein containing the desired peptide sequence is expressed in inclusion bodies and CNBr cleavage is used to release the peptide (Zhang et al., 1998; Piers et al., 1993). This system is not appropriate for high throughput screening due to the labor-intensive aspects of inclusion body isolation and CNBr cleavage. The development of an inducible E. coli expression system that expresses active antimicrobial peptides (under controlled conditions) facilitates high throughput screening of complex recombinant libraries in colonies or microcolonies of cells. By colony, we mean to encompass any clonal population of physically contiguous cells. The definition is intended to encompass archaea, bacteria and eucarya. The definition also is intended to encompass colonies that are visible to the naked eye, as well as microscopic colonies (i.e., “microcolonies”).

Ubiquitin-peptide Fusions

As described above, one of the major challenges in generating a combinatorial library of antimicrobial peptides is to properly express the peptides in the host organism. In one embodiment the invention solves this problem by using ubiquitin as a carrier protein. The 76-amino acid protein ubiquitin is the most conserved eukaryotic protein known, but neither ubiquitin itself nor ubiquitin-specific proteases are present in bacteria (Hershko & Ciechanover, 1992). Ubiquitin fusion proteins occur naturally, and synthetic protein fusions with ubiquitin have been used to increase expression yields of unstable or poorly expressed proteins in E. coli, with the ubiquitin acting as a carrier protein (Butt et al., 1989). The use of ubiquitin fusion technology has several advantages for the production of peptides in E. coli: (1) ubiquitin-peptide fusions are very stable and are expressed at high levels in soluble form (Pilon et al., 1996, 1997); (2) random-sequence peptide libraries cloned as C-terminal fusions with ubiquitin have already been produced with high yields (LaBean et al., 1995); and (3) cleavage of the peptide product from ubiquitin can be performed either in vivo or in vitro by one of several ubiquitin-specific proteases, regardless of the nature of the amino acid immediately following ubiquitin (Gilchrist et al., 1997). Ubiquitin-specific proteases cleave after the C-terminal glycine of ubiquitin, producing the authentic amino-terminus of its fusion partner. The yield-enhancing effect of ubiquitin on its fusion partner is still observed in E. coli during coexpression of a ubiquitin-specific protease and a ubiquitin-protein fusion (Baker et al., 1994).

These properties of ubiquitin fusion technology can be utilized for the expression of antimicrobial peptides in E. coli. Ubiquitin fusions allow screening of peptides directly in colonies, because: (1) the use of ubiquitin as a fusion partner stabilizes expression of the peptide; (2) the ubiquitin-fusion proteins remain soluble, avoiding the production of inclusion bodies that are observed with other fusion partners (Zhang et al., 1998; Callaway et al., 1993; Piers, et al., 1993); and (3) after expression is induced, in vivo cleavage of the inactive fusion protein with ubiquitin-specific proteases regenerates the active antimicrobial peptides with their native amino-termini.

U.S. Pat. No. 5,763,225 (Rechsteiner et al., 1998) and U.S. Pat. No. 5,620,923 (Rechsteiner et al., 1997) describe the synthesis of peptides as ubiquitin fusions. U.S. Pat. No. 5,683,904 (Baker et al., 1997) and U.S. Pat. No. 5,212,058 (Baker et al., 1993) describe a generic class of ubiquitin-specific proteases which specifically cleave at the C-terminus of the ubiquitin moiety in a ubiquitin fusion protein, irrespective of the size of the ubiquitin fusion protein. U.S. Pat. No. 5,847,097 (Bachmair et al., 1998) describes methods of generating desired amino-terminal residues in peptides using a ubiquitin-peptide fusion protein which is specifically cleavable by a ubiquitin-specific endoprotease between the carboxy-terminal residue of ubiquitin and the adjacent amino-terminal residue of the peptide of interest.

Bacterial Viability Assays

The next step in screening a library of antimicrobial peptides comprises a high-throughput assay that is sensitive enough to detect cells that have been killed due to contact with active peptides. This requires the use of one or more viability indicators that are capable of generating an optical signal. By viability indicator, we mean to encompass any compound that can be used to distinguish live cells from dead cells, or any compound that can be used to distinguish cells that are damaged but alive, from cells that are undamaged and alive. An optical signal arising from a viability indicator may indicate that a cell is alive or viable, or alternatively, the signal may indicate that a cell is dead or non-viable. Similarly, the absence of a signal from a viability indicator may be used to determine the state (e.g., dead or alive) of a cell. For example, if a signal from a viability indicator ordinarily indicates that a cell is alive, the absence of a signal from that indicator may be use to determine that a cell is dead. Thus, in one aspect, a viability indicator is a membrane-impermeant compound that may be used to assess the integrity of a cell wall or cell membrane. Fluorescence-based assays have been used extensively for evaluating bacterial viability (Pore, 1994). Many of these assays use nucleic acid stains to differentiate between live and dead cells. Many of these stains show an enhancement in their fluorescence quantum yield after binding to DNA. Such nucleic acid fluorescent stains therefore may be used as viability indicators according to the methods of the present invention. Nucleic acid fluorescent stains include, but are not limited to: Acridine Homodimer, Acridine Orange, 7-Aminoactinomycin D, 9-Amino-6-chloro-2-methoxyacridine, BOBO-1, BOBO-3, BO-PRO-1, BO-POR-3, 4′,6′-Diamidino-2-phenylindole, Dihydroethidium, 4′,6-(Dlimidazolin-2-yl)-2-phenylindole, Ethidium-acridine heterodimer, Ethidium bromide, Ethidium diazide, Ethidium homodimer-1, Ethidium homodimer-2, Ethidium monoazide, Hexidium Iodide, Hoechst 33258, Hoechst 33342,

Hydroxystilamidine methanesulfonate, LDS 751, Oli Green, Pico Green, POPO-1, POPO-3, PO-PRO-1, PO-PRO-3, Propidium Iodide, SYBR Green I, SYBR Green II, SYTO 11 live-cell nucleic acid stain, SYTO 12 live-cell nucleic acid stain, SYTO 13 live-cell nucleic acid stain, SYTO 14 live-cell nucleic acid stain, SYTO 15 live-cell nucleic acid stain, SYTO 16 live-cell nucleic acid stain, SYTO 20 live-cell nucleic acid stain, SYTO 21 live-cell nucleic acid stain, SYTO 22 live-cell nucleic acid stain, SYTO 23 live-cell nucleic acid stain, SYTO 24 live-cell nucleic acid stain, SYTO 25 live-cell nucleic acid stain, SYTO 17 red live-cell nucleic acid stain, SYTOX Green nucleic acid stain, TO-PRO-1, TO-PRO-3, TO-PRO-5, TOTO-1, TOTO-3, YO-PRO-1, YO-PRO-3, YOYO-1; YOYO-3, etc. Additional useful nucleic acid stains are described in the international applications WO 93/06482, DIMERS OF UNSYMMETRICAL CYANINE DYES (published Apr. 1, 1993); U.S. Pat. No. 5,436,134 to Haugland et al., 1995; U.S. Pat. No. 5,321,130 to Yue et al, 1994; U.S. Pat. No. 5,410,030 to Yue et al., 1995; U.S. Pat. No. 5,437,980 to Haugland et al., 1995 and in Haughland, R. P. (2001) Handbook of Fluorescent Probes and Research Chemicals, eighth edition. (Molecular Probes, Eugene, Oreg.). These dyes are available commercially from Molecular Probes (Eugene, Oreg.), Sigma-Aldrich (St. Louis, Mo.) and other chemical suppliers. Methods to detect bacteria and toxins using fluorescent dyes have been described in U.S. Pat. No. 5,994,067 (Wood et al., 1999).

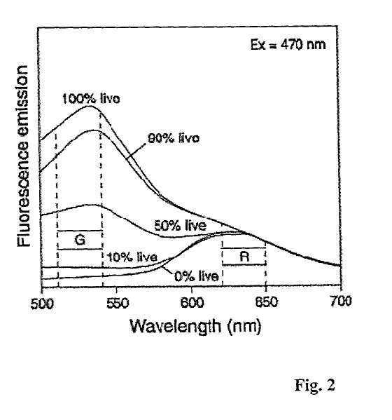

Fluorescence-based live/dead tests are advantageous because: (1) a large assortment of different cell-permeant and cell-impermeant nucleic acid dyes are available that emit fluorescence at different wavelengths; (2) the cells contain large amounts of nucleic acids, and thus signals from the staining reagents are strong; (3) the cells have low intrinsic fluorescence. Many of these stains and assays are commercially available, e.g., the LIVE/DEAD BacLight Bacterial Viability Kit from Molecular Probes (Eugene, Oreg.). The proportion of live and dead E. coli in a bacterial suspension can be determined by measuring the fluorescence properties of stained samples (FIG. 2). Although the use of these fluorescence-based assays is widespread, there have been no reports of their use in high-throughput viability screening of colonies on a support surface.

Loss of the integrity of the bacterial plasma membrane changes the ability of cell-permeant and cell-impermeant stains to label nucleic acids within a cell. The effect of cell-wall directed antibiotics (such as beta-lactams) on membrane integrity can be monitored with cell-impermeant nucleic acid stains such as SYTOX Green (Roth et al., 1997). Since antibacterial peptides are thought to act by disrupting membrane integrity, an assay using these types of nucleic acid stains is useful for monitoring antibacterial peptide activity. The present invention provides a solid-phase antimicrobial peptide assay that enables high throughput screening of antimicrobial peptides expressed in the colonies. Note that it is also possible to employ viability indicators that are not nucleic acid stains or that are not fluorescent. Examples of viability indicators that are fluorescent but do not bind to DNA include carboxyfluorescein diacetate and resazurin (Molecular Probes). Examples of non-fluorescent viability indicators are neutral red and trypan blue (Sigma-Aldrich, St. Louis, Mo.).

Digital Imaging Spectroscopy and Solid-phase Assays for Identifying Antimicrobial Peptides

Digital imaging spectroscopy (DIS) is defined as the combined analysis of both spatial and spectral information so that each picture element (pixel) in a two-dimensional scene includes a third dimension of spectral and/or kinetic information. DIS has been employed in a variety of biological applications ranging from microscopic analysis of Fluorescence Resonance Energy Transfer (FRET) between proteins to macroscopic analyses of bacterial colonies expressing chromogenic enzyme variants (Youvan, 1995; Youvan et al., 1995; Yang et al., 1997; Youvan et al., 1997a,b).

KAIROS has recently developed a MicroColonyImager (MCI) for directed evolution of enzymes by massively parallel screening of combinatorial libraries in microcolonies (Bylina et al., 1999; U.S. Pat. No. 5,914,245). Because of the small size of the microcolonies, they can be grown to high densities on a solid-phase surface (e.g., a 47 mm diameter microporous membrane filter (Poretics; Westborough, Mass.)) and simultaneously imaged by the MCI. Densities as a high as about 10,000 microcolonies per 47 mm disk can be achieved. The microporous membrane filter is one embodiment of a substantially continuous base (i.e., one embodiment of a support surface) that facilitates high-throughput, solid-phase screening.

Cells can be deposited onto the filter by vacuum filtration or by spreading with glass beads. The filter is placed on nutrient medium until colonies form. It can then be transferred to another medium containing an inducer (e.g., isopropyl-beta-D-thiogalactoside, or IPTG) to initiate expression of a cloned gene. If necessary, the colonies can be lysed by exposing them to chloroform vapor. The colonies are contacted with a viability indicator substrate, by means of a saturated wick, to initiate a color-forming reaction. In another embodiment the contacting may be done before the induction step. All the invention requires, in this aspect, is that the viability indicator is present to contact the cells (irrespective of when the indicator has been added, i.e., before or after induction) after induction so that the optical signal arising from the indicator may be used to determine whether the induced gene expression has affected cell viability. The entire filter is then analyzed by the MCI instrument to locate the colonies that have a desired property. The desired colonies are picked and the DNA encoding the gene product from each chosen colony is retrieved. The process of mutagenesis, screening and picking can be repeated until the desired property is obtained. This iterative process is known as ‘directed’ or ‘molecular’ evolution.

In a preferred embodiment of the present invention, the MCI system can be used to monitor differences in fluorescence due to the live/dead viability staining of colonies expressing combinatorial libraries of antimicrobial peptides. The MCI system has previously been employed to acquire kinetics data on microcolonies using exogenous indicator dyes, to identify clones with the desired enzymatic activity, and to recover DNA encoding the corresponding enzyme. In the antimicrobial peptide assays, the MCI's ability to discern spectral differences is important for identifying positive clones.

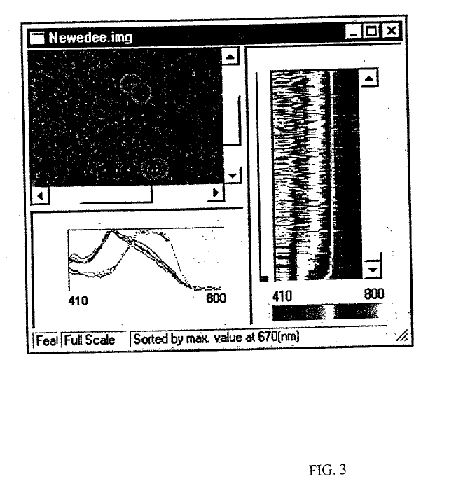

The ability of the MCI to spectrally and spatially distinguish microcolonies based on their production of two different indigoid pigments is illustrated in FIG. 3. A similar fluorogenic live/dead assay procedure (see FIG. 2) is employed as part of the present invention for screening antimicrobial peptide activity. The organization of the MCI graphical user interface (GUI) shown in FIG. 3 can be described as follows: The upper-left window displays a region of interest within a solid phase assay disk (e.g., a 47 mm polycarbonate membrane) bearing E. coli microcolonies. The microcolonies are expressing a library of Agrobacterium faecalis glucosidase (Abg) mutants that have been generated by error-prone mutagenesis. The wild-type glucosidase is capable of hydrolyzing both glucosides and galactosides, but mutagenesis is capable of altering its specificity. The spectral plots of the 15,000 pixels in this window have been sorted in the contour plot on the right. Each row of the contour plot represents a single pixel in the image. The spectrum of each pixel (row) is displayed using a color code rather than a traditional graphical line. The color code instead uses a rainbow, wherein high absorbance is white and low absorbance is black. The contour plot for this example covers the spectral range from 410 nm (on the left) to 800 nm (on the right). As evidenced from the contour plot's scrollbar, only a small number of the 15,000 pixel-spectra are shown. Pixels representing regions in which Red-gal is preferentially cleaved are sorted to the bottom of the contour plot. The actual spectra that are plotted are indicated by color-encoded tic marks to the lower left of the contour plot. The conventional spectral plots in the lower left window show the spectra of single pixels of a microcolony in which Red-gal is preferentially cleaved. In this microcolony, the predominant indigo product absorbs maximally at 540 nm. These pixels are color-coded red in the upper-left window. For clarity, they have also been circled in red to identify this single microcolony in which galactosidase activity predominates. The spectral plots in the lower left window also show the spectra of pixels that preferentially cleave X-glu. In these microcolonies, the indigo product absorbs maximally at 615 nm. These pixels are color-coded green in the upper-left window and identify microcolonies in which the glucosidase activity has not been altered. The MCI can also obtain kinetics data for the enzymatic reactions occurring in the microcolonies. In this mode, the spectral window in the lower left corner of the GUI is replaced by a kinetics window.

REM and Cassette Design

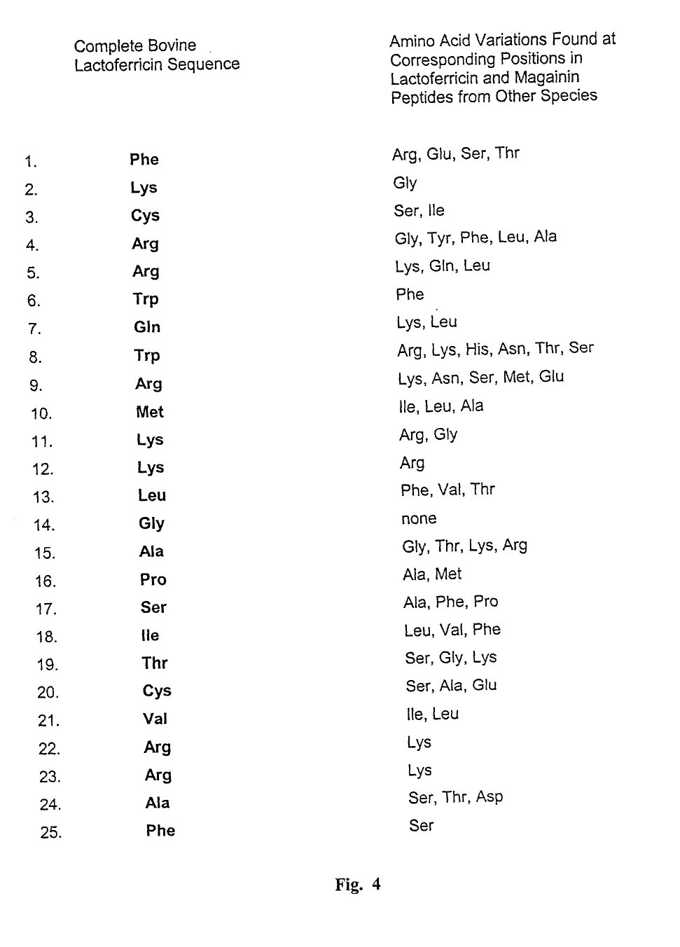

The first step in the synthesis of a combinatorial library of antimicrobial peptides is the design of the peptide cassette. A library of REM-based double-stranded oligonucleotides is synthesized and assembled based on the methods described in Delagrave et al. (1993). The sequences in this library are then cloned and expressed as ubiquitin fusions in E. coli. The results obtained after screening the initial combinatorial library are then used to continue the REM process. As is the case of all combinatorial mutagenesis experiments, the number of possible sequences grows exponentially with the number of residues mutagenized. In one embodiment, the initial combinatorial cassette introduces a highly complex library by doping at several different positions. This may generate a highly diverse library with a complexity at the protein level of approximately 10⁶ or more. Alternatively, the cassette can be designed according to a variety of heuristic principles that either increase or decrease the restrictions initially set on the combinatorial library. In this alternative embodiment, sequence alignments among homologous antimicrobial peptides are used for cassette design. For example, the alignment of sequences from a few lactoferricin and magainin species (Odell et al, 1996, Maloy & Kari, 1995) in FIG. 4 can be used to construct a combinatorial lactoferricin cassette.

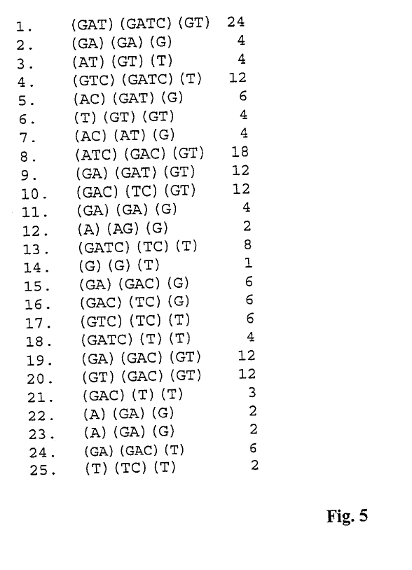

Using the program CyberDope (Arkin & Youvan, 1992a) and two alternative fitting functions, optimized combinatorial doping patterns can be determined for each of the 25 sequence positions shown in FIG. 4. These ‘targeted’ libraries enable one to more efficiently search sequence space. Even so, the overall sequence complexity of a combinatorial cassette encoding the entire peptide would be 10¹⁸; therefore, it may be advantageous to divide the peptide into two or more segments and then to re-optimize using the EEM strategy. Alternatively, NN(GT) triplets can be used at certain positions to increase the complexity still further, requiring four cassettes of sequence complexity ˜32⁶ or ˜10⁹ as the bases of the EEM experiment. An extensive discussion and examples of REM and EEM experiments can be found at http://www.kairos-scientific.com/searchable/cyberdope.html (a copy of which is appended t the end of this specification) and in several journal articles (Arkin & Youvan, 1992b; Goldman & Youvan, 1992; Youvan et al., 1992; Delagrave & Youvan, 1993; Delagrave et al., 1993; Goldman et al., 1994; Goldman & Youvan, 1995; Delagrave et al., 1995). FIG. 5 shows the results of CyberDope calculations on the sequence variations for each position displayed in FIG. 4.

Constructing Ubiquitin Fusions

To facilitate expression of ubiquitin fusions, we constructed a synthetic ubiquitin gene for optimized fusions with ‘partner’ proteins within the high-level expression plasmid pQE-70 (Qiagen, Valencia, Calif.). Expression of a gene requires having an RNA-encoding DNA sequence that is operably linked to a promoter sequence. This means that the DNA sequence is therefore capable of producing corresponding RNA transcripts when the promoter is recognized by a suitable polymerase. Although the gene can be expressed from a DNA sequence incorporated into the host chromosome, the preferred embodiment is to use a plasmid or other vector for expression. Expression in this vector is under the control of two lac operator sequences, allowing tight, efficient repression of the powerful T5 promoter in the absence of the inducer, IPTG. In order to produce ample repressor in the expression strain M15, plasmid, pREP4 (containing the lac I gene) constitutively expresses the lac repressor protein. This ubiquitin expression system is similar to that used by Pilon et al. (1996, 1997). Silent in-frame Afl II and Sac II sites within the 3′-coding end of the ubiquitin gene were incorporated to facilitate cloning of peptides and proteins as fusions to the C-terminus of ubiquitin. We have successfully used this system to express proteins that would otherwise form inclusion bodies in E. coli. These experiments are consistent with previous studies that indicate that ubiquitin-peptide/protein fusions are very stable and are expressed at high levels in soluble form (Pilon et al., 1996). We have also used ubiquitin-GFP fusions to establish pre-induction growth conditions that better repress the background expression of the fusion protein. Background expression of the ubiquitin-GFP fusion was measured by monitoring fluorescence levels in growing colonies. Low levels of fluorescence were observed when colonies were grown on LB plates lacking inducer. Addition of glucose to the LB plates greatly suppressed this background fluorescence. These ‘repressing’ growth conditions can be used with the clones containing antimicrobial peptides fused to ubiquitin. The plasmids and procedures applicable to the cloning and expression of antimicrobial peptides fused to ubiquitin are described in detail in the following sections.

Antimicrobial Peptides Expressed as Ubiquitin Fusions

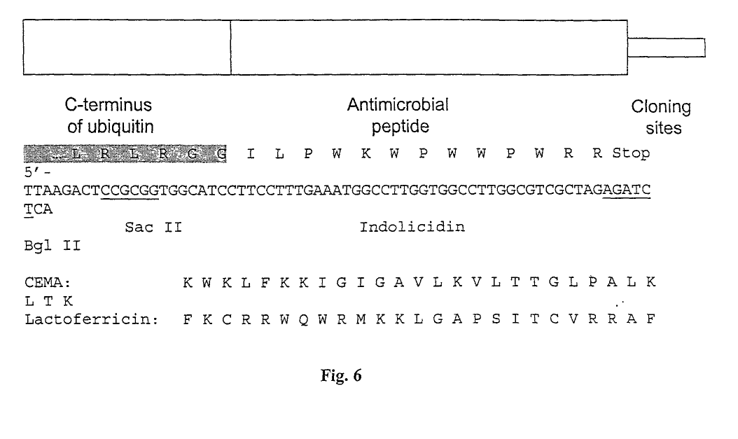

A useful method for expressing antimicrobial peptides as C-terminal fusions with ubiquitin is described herein (FIG. 6). The peptides may comprise the alphahelical peptide CEMA, the extended peptide indolicidin, and the looped peptide lactoferricin. Indolicidin and CEMA have previously been expressed as fusion proteins in inclusion bodies (Zhang et al., 1998). While the disulfide bond present in lactoferricin will not be formed during E. coli expression, it has been shown that this bond is not required for antibacterial activity of the peptide (Bellamy et al., 1992).

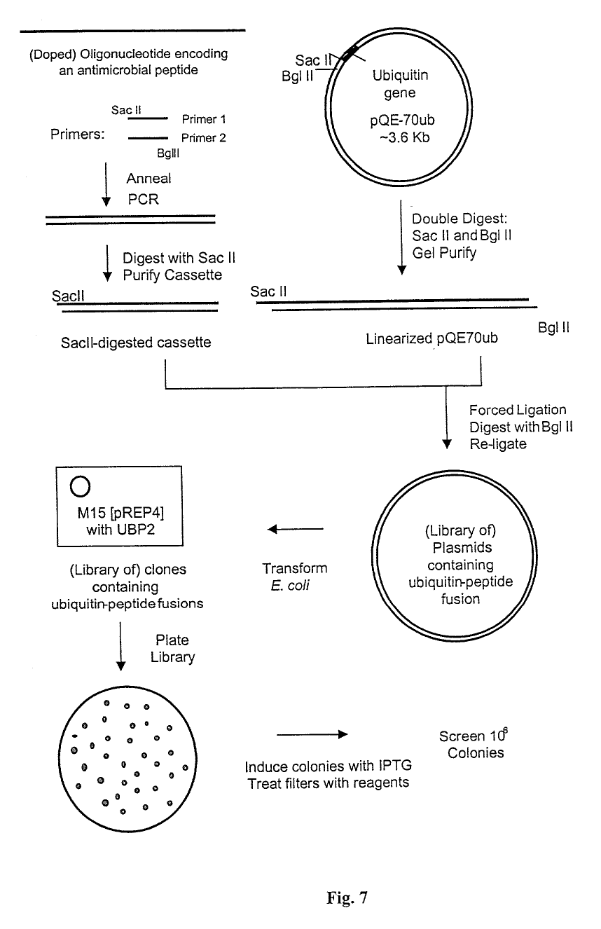

After the DNA fragment is purified, it is cloned into the pQE70ub vector as outlined in FIG. 7. Ligation reactions are driven to completion, without creating concatemers, by establishing pseudo first-order and unimolecular conditions in two separate steps: First, only one end of the double-stranded DNA fragment is cut prior to ligating it with the vector (at high concentrations). Next, the partially ligated construct is cut with the second enzyme and then diluted to avoid any further bimolecular reactions. The plasmid is then closed by an efficient unimolecular ligation. The E. coli strain M15 [pREP4] (Qiagen) can be used as the host for these ubiquitin fusion constructs. The plasmid pREP4 contains a copy of the lac I gene that constitutively expresses the lac repressor protein, so that the ubiquitin fusion protein can only be expressed upon induction with IPTG. Expression of the soluble inactive fusion protein can be confirmed by SDS-PAGE.

For expression of the active free peptide, the ubiquitin-specific protease UBP2 can be coexpressed with the ubiquitin-fusion protein (Baker et al., 1994). The expression host is modified by cloning the UBP2 gene (Baker et al., 1992) into the pREP4 plasmid. Optical screening of colonies is performed using the live/dead solid phase assay described below. Colonies containing both the ubiquitin-peptide fusion and the ubiquitin protease genes are grown on filters under conditions that repress production of the proteins. Once the colonies reach the appropriate size, the filters are exposed to IPTG to induce expression of the antimicrobial peptides and treated with nucleic acid stains (i.e., viability indicators) to measure cell death. Colonies that contain peptides which kill cells are identified using the MicroColonyImager. Filters are imaged to determine the fluorescence signal from each colony on the filter. Colonies expressing positive candidates are picked, and the DNA is retrieved and sequenced. In a preferred embodiment, the DNA is typically retrieved by resuspending the picked colony in buffer, re-transforming an appropriate host strain by electroporation, plating the transformants, and picking a clone with the confirmed phenotype. Plasmid DNA isolated from this clone is then used for sequencing. Alternatively, the plasmid insert from the picked colony is amplified by PCR and the resulting PCR product is sequenced. Although DNA retrieval might also comprise the steps of extracting mRNA from the positive colonies and sequencing them by means of RT-PCR (Ausubel, 2000), such an approach is not a preferred embodiment of the present invention.

The MicroColonyImager can also be used to measure the changes in fluorescence signal over time. This kinetic information indicates how quickly cells in the colonies are being permeabilized by the antibiotic—an indication of the activity of the peptide. IPTG induction levels can be modified to alter these kinetics. For the differentiation of very active peptides, lower levels of induction may provide better kinetic resolution of differences in antimicrobial activities.

Although one might assume that most of the peptides will be inactive when fused to ubiquitin, this may not be the case for the most active anti-microbial peptide sequences that can be generated. The screening system described in the present invention can identify these high activity peptides, regardless of whether they are still fused to ubiquitin. Colonies containing the gene for very active antimicrobial peptides will grow on the filters, since the ubiquitin fusion protein (whether active or inactive) is only expressed under induction conditions. Pre-induction growth conditions that substantially repress background expression of protein for this expression system have been established by using GFP-ubiquitin fusions (see above).

Examples of Other Carrier Proteins for Expressing Peptides

Other, somewhat less preferred embodiments for expressing peptide fusions can be employed (LaVallie & McCoy, 1995). For example, the peptide can be expressed as a fusion to the maltose binding protein by cloning the targeted oligonucleotide library into the pMAL vector downstream of the mal E gene and transforming E. coli host TB1 with the resulting library of plasmid constructs. The putative antimicrobial peptide can be cleaved from the carrier, if desired, by a sequence-specific protease. The pMAL Protein Fusion and Purification System is available as a kit from New England BioLabs (Beverly, Mass.).

In addition, there are other methods for cell surface display and phage display of the peptide library. Cell surface display uses a protein expressed on the outer membrane surface of a cell to provide a ‘vehicle’ for carrying a peptide or small protein. Surface display systems have been described for gram-negative and gram-positive bacteria (Francisco & Georgiou, 1994; Stahl & Uhlen, 1997; Georgiou et al., 1997; Chang et al., 1999) and for yeast (Boder & Wittrup, 1997; Cereghino & Cregg, 1999). Peptides or proteins can be fused to the C-terminus or N-terminus of the carrier protein with a short linker peptide. The linker may additionally contain a specific recognition sequence for an endopeptidase, such as Factor Xa (Nagai & Thogerson, 1984) or enterokinase (Collins-Racie et al., 1995). Treatment of the cells expressing the peptide with the appropriate peptidase thus permits release of the peptide from the cell surface (LaVallie et al., 1994). The recognition sequence can be selected so that only the authentic peptide will be released.

In one such embodiment of the present invention, the REM cassette containing the antimicrobial peptide library is cloned into a plasmid construct that contains the chimeric Lpp-OmpA display protein (Francisco et al., 1992). Lpp is the major E. coli lipoprotein, and OmpA is an E. coli outer membrane protein. The fragment used in the present method comprises an N-terminal signal sequence (20 residues) and the first 9 residues of the mature Lpp protein. The OmpA fragment comprises a segment of 114 amino acids (residues 46-159) which is fused to the C-terminal end of the Lpp fragment by a Gly-Ile dipeptide linker. The first codon of the REM cassette is attached in frame to the 3′-end of the OmpA fragment by a DNA sequence encoding another short linker. This allows the peptide library to be expressed on the C-terminus of the carrier. The linker peptide contains a flexible region (such as Gly-Gly-Gly-Ser) (SEQ ID NO: 1) followed by a recognition sequence for a sequence-specific endopeptidase, such as enterokinase (Asp-Asp-Asp-Asp-Lys) (SEQ ID NO: 2). E. coli strain JM109 is a suitable host strain for expression of the library. After the library is expressed in colonies on a membrane filter, the filter can be treated with a solution of enterokinase to release the peptides. Enterokinase is available from Novagen (Madison, Wis.), Stratagene (La Jolla, Calif.) and other suppliers. A filter containing a confluent lawn of target cells is then placed over the peptide-containing filter, and the viability assay is performed as described in the next section. Colonies expressing positive candidates are picked, and the DNA is retrieved and sequenced.

In another embodiment, the peptide library is expressed by means of phage display. In this system, the peptide library can be expressed at the C-terminus of a bacteriophage coat protein (Scott & Smith, 1990; Wells & Lowman, 1992; Hill & Stockley, 1996; Rodi & Makowski, 1999). The peptide is expressed on the outside of the virus particle, while the corresponding DNA encoding the peptide is inside. The T7Select Phage Display System from Novagen (Madison, Wis.) is useful for this type of expression. A DNA sequence encoding the linker peptide Asn-Ser-Gly-Gly-Gly-Ser-Asp-Asp-Asp-Asp-Lys (SEQ ID NO: 3) (containing a 5′ EcoR I site in the DNA sequence and an enterokinase cleavage site at the carboxy-terminus of the Lys residue in the peptide sequence) is included on the 5′-end of the REM cassette. A short sequence containing a Hind III site is attached after the stop codon on the 3′-end. These two restriction sites facilitate cloning of the REM cassette into a T7Select vector (e.g., the T7Select 415-1b vector). The host E. coli (e.g., strain BL21) is transformed with the vector library and plated to form a lawn of phage plaques at less than confluent density. The lawn of phage plaques is preferably grown on a microporous filter membrane so that it can be removed from the nutrient agar for later assay. The peptides are then released from the phage surface by treatment with enterokinase. A filter containing a confluent lawn of target cells is then placed over the peptide-containing filter, and a viability assay is performed as described in the next section. Plaques expressing positive candidates are picked, and the DNA is retrieved and sequenced.

Construction of High-Complexity Peptide Libraries

When a known antimicrobial sequence is chosen as a starting or ‘parental’ sequence for phylogenetic doping, it is preferable to confirm that the given antimicrobial peptide can be properly expressed as a ubiquitin fusion. A high complexity peptide library can then be constructed by ‘doping’ the sequence and expressing the library. Doped oligonucleotides that encode these libraries can be designed as described above in the ‘REM and Cassette Design’ section. In one embodiment, doped oligonucleotides are copied with polymerase using a suitably complementary DNA primer, rather than being hybridized with a synthetic strand, to avoid mismatched areas in the DNA. The resulting DNA fragments are cloned into the pQE70ub vector (or another suitable vector) as described in FIG. 7. Experimental complexities of approximately 10⁶ mutants per library can be routinely obtained with this method. In one embodiment, E. coli cells that are expressing the peptide library also comprise the target cells for the activity assay. In this case, intracellular expression of an active peptide will kill the cells within the colony.

In another embodiment, E. coli colonies that express the peptide library are used only for the purpose of generating the peptides. The target cells, which actually indicate the effect of the peptides on cell viability, comprise a lawn of cells such as bacteria on a second membrane filter. These target cells are placed in contact with the expressing cells and indicate which colonies are producing an active peptide. The target cells may comprise E. coli or another pathogenic organism. Expression of the peptide libraries in E. coli is highly advantageous for the genetic screening system because it generates libraries having the very high complexities that are needed to search sequence space. However, high-throughput assays for screening candidate antimicrobial peptides against other organisms, such as well-known pathogens, are of great value. This type of ‘sandwich’ viability assay is described below.

In a slightly different embodiment, the antimicrobial molecule produced by the colonies can be a small molecule (e.g., having a mass of less than about 1,000 daltons) that is not a peptide or that contains only a few peptide bonds. For example, antibiotics such as polyketides are known to be produced by complex multi-enzyme pathways. Many of the synthetic genes for these pathways have been cloned for the purpose of performing metabolic engineering to create novel antibiotic products (Leadlay, 1997; Tsoi & Khosla, 1995; Fu & Khosla, 1996; McDaniel et al., 1999; Xue et al., 1999). Some of these methods involve genetically recombining genes that encode for enzymes in these pathways, a process termed ‘combinatorial biosynthesis’. Deletions and mutations can also be incorporated into the genes. This type of directed evolution creates new enzyme pathways that in turn produce new antibiotic substances. Colonies of cells expressing libraries of these various gene combinations can be screened using the assay methods described in the Examples below.

DNA Retrieval and Throughput

Optical screening of libraries can be performed using the MicroColonyImager with the live/dead solid-phase assays described herein. Once colonies or microcolonies of interest are identified (i.e., those that express a candidate antimicrobial molecule), a tiny portion of the disk containing the ‘positive’ colony is cored either manually or robotically to pick the colony off the filter surface. Alternatively, a positive colony can be picked off the membrane with a pipette tip. Recovered DNA is eluted from the filter fragment and used to electroporate competent E. coli. The resulting transformants are re-assayed to identify and confirm the desired antimicrobial peptide variant and to purify it away from any other variants that may have been carried over during recovery of the colony. Positive clones are characterized by DNA sequencing. The sequence information obtained is used to design a cassette for subsequent rounds of REM experiments. If approximately 90 mm diameter filter membranes are employed (with approximately 40,000 microcolonies per filter), one technician can stagger production and analysis of sets of filters, such that overall throughput is approximately 1,000,000 colonies per day. The colony density per filter can also be increased in order to increase the throughput.

### EXAMPLE 1

### A Solid Phase Colony Viability Assay

The high-throughput solid-phase viability assay is demonstrated here using the non-permeant SYTOX Green fluorescent stain (Molecular Probes, Eugene, Oreg.). The SYTOX green acts as a viability indicator for monitoring the metabolic status and permeability of the cells. In this example, cells in microcolonies grown on filter membranes are selectively labeled. An advantage of using this type of fluorescent dye is that the fluorescence emission from the unbound dye molecules is very low, thus reducing the intensity of the background signal. This also means that the filter does not need to be washed to remove fluorescence contributions from unbound dye.

An ampicillin-based model screening system is used to test the SYTOX Green staining: microcolonies that are either ampicillin-sensitive [the kanamycin-resistant E. coli strain M15 [pREP4] (QIAgen)] or ampicillin-resistant (M15 [pREP4] bearing pLITMUS28) are first exposed to ampicillin (100 microgram/ml) and then treated with 5 micromolar SYTOX Green. Ampicillin is expected to disrupt cell wall synthesis in the sensitive microcolonies, allowing permeabilization of their cells, and resulting in staining of their DNA with the SYTOX Green. Ampicillin-resistant microcolonies are not stained by SYTOX Green because the cells in these microcolonies produce the enzyme beta-lactamase, which inactivates the antibiotic. Filters covered with either ampicillin-resistant microcolonies or ampicillin-sensitive microcolonies are prepared. After the cells are deposited on filter membranes, the filters are transferred to LB (Luria-Bertani nutrient agar) plates containing 25 microgram/ml kanamycin and incubated until microcolonies appear on the filters. The filters are then transferred to LB plates containing 100 microgram/ml ampicillin and incubated for 60 minutes. After this incubation, the filters are transferred to LB agarose plates containing 5 micromolar SYTOX Green. The agar plate acts as a wick to facilitate contacting the cells with the viability indicator. Many different materials can be used as a wick, including suspensions of agar, agarose, acrylamide or other gel-forming materials, as well as filter paper saturated with buffered indicator solution. The use of a wick is described in U.S. Pat. No. 5,914,245. The MicroColonyImager (MCI) is used to monitor the fluorescence emission of the microcolonies on the filter using a 510 nm long pass filter. Ampicillin-sensitive colonies emit fluorescence while ampicillin-resistant colonies do not.

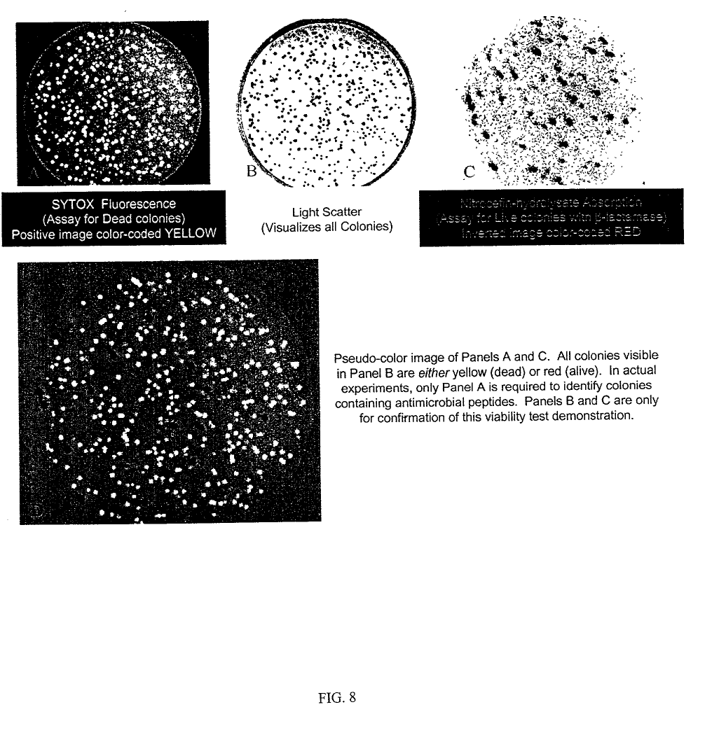

This SYTOX Green colony viability assay was used to screen an E. coli library of mutagenized beta-lactamase genes (FIG. 8). The beta-lactamase gene from pUC18 was cloned into a kanamycin-resistant pET28 derivative (Novagen, Madison, Wis.) and error-prone PCR was used to heavily mutagenize the gene. After a portion of the resulting mutagenized library was deposited on a filter, the filter was transferred to LB plates containing kanamycin and incubated until microcolonies appeared on the filters. The filter was then exposed to 100 microgram/ml ampicillin and treated with 5 micromolar SYTOX Green. The MicroColonyImager (MCI) was used here to monitor the fluorescence emission from microcolonies on the filter using 500 nm excitation and a 510 nm long pass filter for the emission. After this imaging, the chromogenic beta-lactamase substrate nitrocefin was used to independently confirm which colonies on the filter possessed beta-lactamase activity. A second wick comprising a disk of Whatman paper saturated with 500 microgram/ml nitrocefin was overlayed on the filter and the MCI device was used to monitor absorption of nitrocefin hydrolysis at about 550 nm. Microcolonies which catalyzed nitrocefin hydrolysis were not stained with SYTOX Green, and fluorescent microcolonies did not catalyze nitrocefin hydrolysis. Other nucleic acid stains can also be used for this viability assay. These include permeant SYTO blue, SYTO green and SYTO red from Molecular Probes, as well the non-permeant stain propidium iodide.

It should be noted that in the example shown in FIG. 8, the chromogenic beta-lactamase substrate nitrocefin was used to independently confirm which colonies on the filter possessed beta-lactamase activity. In an alternative embodiment, sensitive and resistant strains are selected so that the live/dead assay is independently confirmed by a second enzyme assay. For example, X-Gal (5-Bromo-4-chloro-3-indolyl-beta-D-galactoside) dependent indigo formation can be used to differentiate lac⁻ and lac⁺ phenotypes that are markers for antibiotic sensitive (M15[pREP4]) and resistant (M15[pREP4] bearing pLITMUS28) colonies, respectively.

This optical assay can be used to screen a number of different types of libraries of antimicrobial compounds for activity, including the unconstrained REM peptide libraries described above, as well as the constrained peptide aptamer libraries described in Mekalanos and Blum (WO 99/50462). The measurement may also utilize various formats, including the direct assay format described here (in which the colonies of the host organism expressing the peptides are killed) or the indirect (‘sandwich’) assay format, which is described in the next Example. The MicroColonyImager can also be used to measure the changes in fluorescence signal over time. This kinetic information indicates how rapidly cells in the colonies become permeabilized through the activity of the antimicrobial compounds. Kinetic information obtained from antimicrobial peptide libraries can be used to identify colonies of the filter containing the most active peptides.

Optimization of a Solid Phase Viability Assay

For any suitable pair of permeant and non-permeant nucleic acid stains (i.e., ones that provide the greatest differentiation in fluorescent signals between colonies on a filter membrane containing live and dead cells), other assay conditions can be optimized. These parameters for optimization may include the composition of the growth medium, growth conditions, filter materials and stain delivery methods. These parameters can be adjusted to minimize heterogeneity among colonies and maximize live cell percentage within ‘live’ colonies.

### EXAMPLE 2

### A ‘Sandwich’ Assay

The screening assay of the present invention can also be adapted to accommodate the embodiment described above wherein the peptide expression library is on one filter and the target cells are on another filter. This type of indirect ‘sandwich’ assay incorporates elements of earlier low-throughput techniques previously described in the literature. For example, agar-based methods that detect growth inhibition of different organisms have been reported (Westerhoff et al., 1995; Helmerhorst et al., 1997). This type of assay is carried out according to the following protocol. First, a culture is plated on agar to grow confluently. Antimicrobial peptide samples are then spotted on each agar plate and screened for zones of growth inhibition. However, in the high-throughput screening assay of the present invention, instead of spotting samples of antimicrobial peptides onto the agar, a large library of peptide-expressing microcolonies is brought into contact with a lawn of target organisms. These target organisms may comprise pathogens. This is accomplished by overlaying the ‘target’ filter onto the ‘expression’ filter containing lysed colonies. Microcolonies on the expression filter can be lysed with chloroform vapor prior to contact with the confluent lawn, and this pre-treatment releases peptides from all the microcolonies (whether active on E. coli or not). During incubation, the MCI device may be used to identify areas on a target filter (i.e., within the confluent lawn) that are affected by the peptide products of particular microcolonies on the filter underneath it. This effect is detected either by identifying clearing zones within the lawn, or by using dyes that differentially stain live and dead cells in the lawn). Once region(s) of activity are identified, a small portion of the filter containing the corresponding ‘positive’ microcolony is retrieved. E. coli are then transformed with the DNA eluted from the filter fragment. The transformants are re-purified, colonies are picked and confirmed as positives, and their plasmid DNA is isolated. The sequence of the antimicrobial peptide gene can then be determined. The activity of the most promising peptides can also be confirmed by retesting a battery of target bacterial pathogens using a modification of the standardized method for susceptibility testing (Steinberg, et al, 1997).

Due to fact that the expressing colonies are lysed, the expression filter will become highly fluorescent when placed in contact with a fluorogenic viability indicator on the wick underneath it. Therefore it is advantageous to use ‘black’ filter membranes for both the expression and target cells and to excite the filters in an epifluorescence configuration. Black polyester track-etch membranes are available from Poretics (Westborough, Mass.). It is also advantageous to invert the expression membrane so that the colonies are facing down toward the wick rather than up toward the target membrane. This orientation suppresses excitation of the fluorescent material in the expressing colonies due to the presence of the intervening filter, while simultaneously allowing diffusion of the peptides (through the back of the expression membrane) upward to the underside of the membrane containing the target cells.

In the embodiment wherein the peptide library is expressed using phage display, it is also advantageous to suppress the fluorescence signal from the lysed cells on the expression lawn. As in the previous example, it is useful to employ black filter membranes and invert the expression filter for the assay. An epifluorescence configuration as described in U.S. Pat. No. 5,914,245 is preferred.

### EXAMPLE 3

### Liquid-phase Screening of Combinatorial Libraries

In another, less preferred embodiment, vectors containing REM libraries are transformed into a host (e.g., E. coli) for expression as described above, but screening and retrieval of positive cells are performed using a microplate assay or fluorescence activated cell sorting (FACS). Preferably, the REM library is expressed as a fusion to a carrier protein (e.g., ubiquitin) and the gene for the enzyme that is required for releasing the peptide from the carrier protein (e.g., a ubiquitin-specific protease) is co-expressed in the same host. Microplate assays can be performed by picking colonies expressing the peptides from an induction plate and transferring each colony to a well of a microplate or similar multi-well apparatus. Fluorogenic or chromogenic dyes that indicate viability are then added to each well. The viability of the cells in each well can be read photometrically and compared to a well containing control cells. DNA is retrieved from the ‘positive’ wells and sequenced. In the FACS assay, the host cells are transformed and then expression is induced in liquid medium. The cells are then centrifuged and resuspended in buffer containing at least one fluorogenic viability indicator. Methods for measuring the viability of individual cells using flow cytometry are well known in the art (Pore, 1994; Suller & Lloyd, 1999; Porter et al., 1996; Roth et al., 1997; Caron et al., 1998; Chapple et al., 1998a,b; Gottfredsson et al., 1998; Swarts et al., 1998; Mortimer et al., 2000). A pool of DNA retrieved from the positive candidates can then be amplified by PCR (if necessary), cloned, and sequenced.

### EXAMPLE 4

Screening Libraries without Biological Expression

In an alternative embodiment, the peptide or chemical library is created artificially (synthetically) rather than biosynthetically to create a synthetic solid-phase array. For example, the members of the library can be synthesized on a peptide synthesizer and then spotted onto a polymeric membrane or a glass or plastic surface. In one embodiment, the array is positionally encoded, so that a given x,y-position of a spot corresponds to a particular member of the library. A membrane containing a layer of target cells is then overlaid on this array and assayed as described above. The chemical structure of the peptide or other candidate molecule in any active spot can then be determined by noting the position of the spot in the array, or by picking the spot. The chemical identity or sequence of the molecule comprising that member of the library is then determined. Alternatively, the peptide or small molecule library can be directly synthesized on beads (or other solid-phase material) using combinatorial chemistry and then assayed for antibiotic activity (Mata, 1999; Tong & Nielsen, 1996). Beads that are in contact with killing zones on the target filter are then retrieved and analyzed.

### EXAMPLE 5

### Screening Libraries Using Other Types of Viability Indicators

In addition to assays incorporating nucleic acid fluorescent stains, a wide range of optical assays can be used to monitor the viability or permeability of cells in colonies and microcolonies. Colonies containing cells which express particular enzymes, such as glucosidases and esterases, can be exposed to cell-impermeant substrates for that enzyme. Only colonies containing cells with compromised membranes will take up the substrate to react with said enzyme. For example, it is known that E. coli colonies expressing a beta-glucosidase will not turn blue in the presence of X-Glu (5-Bromo-4-chloro-3-indolyl-beta-D-glucopyranoside), but these same colonies will turn blue in the presence of X-Glu if first lysed with chloroform vapor. Other enzyme-substrate pairs are well known in the art. Potentiometric dyes can be used to detect transmembrane potential gradients to differentiate between live and dead cells. In addition, pH indicators can be used. For example, fluorescence differences between live and dead colonies can be measured if the cells express a green fluorescent protein (GFP) derivative whose fluorescent properties are pH-sensitive (for example, see Robey et al., 1998). If the colonies that express the peptide library (or are in contact with it) are exposed to a buffered solution at a pH which is lower or higher pH than the internal pH of a living cell, the pH inside cells of live colonies will not change, while the pH inside cells of dead colonies will equilibrate to that of the buffered solution. Colonies containing dead or permeabilized cells will indicate the pH induced changes via the fluorescence of the expressed GFP.

### REFERENCES

Arkin, A. P. & Youvan, D. C. (1992a) Optimizing nucleotide mixtures to encode specific subsets of amino acids for semi-random mutagenesis. Biotechnology (NY) 10:297-300.

Arkin, A. P. & Youvan, D. C. (1992b) An algorithm for protein engineering: simulations of recursive ensemble mutagenesis. Proc. Natl. Acad. Sci. U.S.A. 89:7811-7815.

Arkin, A., Goldman, E., Robles, S., Coleman, W., Goddard, C., Yang, M. & Youvan, D. C. (1990) Applications of imaging spectroscopy in molecular biology. II. Colony screening based on absorption spectra. Biotechnology (NY) 8:746-749.

Ausubel, F. M., Brent, R., Kingston, R. E., Moore, D. D., Seidman, J. G., Smith, J. A. & Struhl, K., eds. (2000) Current Protocols in Molecular Biology, Wiley, N.Y.

Baker, R., Tobias, J. W. & Varshavsky, A. (1992) Ubiquitin-specific proteases of Saccharomyces cerevisiae. Cloning of UBP2 and UBP3, and functional analysis of the UBP gene family. J Biol. Chem. 267:23364-23375.

Baker, R., Smith, S., Marano, R., McKee, J. & Board, P. (1994) Protein expression using cotranslational fusion and cleavage of ubiquitin. Mutagenesis of the glutathione-binding site of human Pi class glutathione S-transferase. J Biol. Chem. 269:25381-25386.

Bellamy, W., Takase, M., Yamauchi, K., Wakabayashi, H., Kawase, K. & Tomita, M. (1992) Identification of the bactericidal domain of lactoferrin. Biochim. Biophys. Acta 1121:130-136.

Birren, B., Green, E. D., Klapholz, S., Myers, R. M., Riethman, H. & Roskams, J., eds. (1999) Genome Analysis, A Laboratory Manual, Vol. 3, Cloning Systems, Cold Spring Harbor Laboratory Press, Plainview, N.Y.

Blondelle, S. E., Takahashi, E., Houghten, R. & Perez-Paya, E. (1996) Rapid identification of compounds with enhanced antimicrobial activity by using conformationally defined combinatorial libraries. Biochem. J. 313:141-147.

Blum, J. H., Dove, S. L., Hochschild, A. & Mekalanos, J. J. (2000) Isolation of peptide aptamers that inhibit intracellular processes. Proc. Natl. Acad. Sci. U.S.A. 97:2241-2246.

Boder, E. T. & Wittrup, K. D. (1997) Yeast surface display for screening combinatorial polypeptide libraries. Nat. Biotechnol. 15:553-557.

Boman, H. G. (1995) Peptide antibiotics and their role in innate immunity. Annu. Rev. Immunol. 13:61-92.

Butt, T. R., Jonnalagadda, S., Monia, B. P., Sternberg, E. J., Marsh, J. A., Stadel, J. M., Ecker, D. J. & Crooke, S. T. (1989) Ubiquitin fusion augments the yield of cloned gene products in Escherichia coli. Proc. Natl. Acad. Sci. U.S.A. 86:2540-2544.

Bylina, E. J., Coleman, W. J., Dilworth, M. R., Silva, C. M., Yang, M. M. & Youvan, D. C. (1999) Solid phase enzyme kinetics screening in microcolonies. U.S. Pat. No. 5,914,245.

Callaway, J. E., Lai, J., Haselbeck, B., Baltaian, M., Bonnesen, S. P., Weickmann, J., Wilcox, G. & Lei, S. P. (1993) Modification of the C terminus of cecropin is essential for broad-spectrum antimicrobial activity. Antimicrob. Agents Chemother. 37:1614-1619.

Caron, C. N., Stephens, P. & Bradley, R. A. (1998) Assessment of bacterial viability status by flow cytometry and single cell sorting. J. Appl. Microbiol. 84:988-998.

Cereghino, G. P. & Cregg, J. M. (1999) Applications of yeast in biotechnology: protein production and genetic analysis. Curr. Opin. Biotechnol. 10:422-427.

Chang, H. J., Sheu, S. Y. & Lo, S. J. (1999) Expression of foreign antigens on the surface of Escherichia coli by fusion to the outer membrane protein traT. J. Biomed. Sci. 6:64-70.

Chapple, D. S., Joannou, C. L., Mason, D. J., Shergill, J. K., Odell, E. W., Gant, V. & Evans, R. W. (1998a) A helical region on human lactoferrin. Its role in antibacterial pathogenesis. Adv. Exp. Med. Biol. 443:215-220.

Chapple, D. S., Mason, D. J., Joannou, C. L., Odell, E. W., Gant, V. & Evans, R. W. (1998b) Structure-function relationship of antibacterial synthetic peptides homologous to a helical surface region on human lactoferrin against Escherichia coli serotype Olll. Infect. Immun. 66:2434-2440.

Collins-Racie, L. A., McColgan, J. M., Grant, K. L., DiBlasio-Smith, E. A., McCoy, J. M. & LaVallie, E. R. (1995) Production of recombinant bovine enterokinase catalytic subunit in Escherichia coli using the novel secretory fusion partner DsbA. Biotechnology (NY) 13:982-987.

Darveau, R. P., Cunningham, M. D., Seachord, C. L., Cassiano-Clough, L., Cosand, W. L., Blake, J. & Watkins, C. S. (1991) Beta-lactam antibiotics potentiate magainin 2 antimicrobial activity in vitro and in vivo. Antimicrob. Agents Chemother. 35:1153-1159.

Delagrave, S. & Youvan, D. C. (1993) Searching sequence space to engineer proteins: exponential ensemble mutagenesis. Biotechnology (NY) 11:1548-1552.

Delagrave, S., Goldman, E. R. & Youvan, D. C. (1993) Recursive ensemble mutagenesis. Protein Eng. 6:327-331

Delagrave, S., Goldman, E. & Youvan, D. C. (1995) Context dependence of phenotype prediction and diversity in combinatorial mutagenesis. Protein Eng. 8:237-242.

Domin, M. A. (1998) Highly virulent pathogens—a post antibiotic era? Br. J. Theatre Nurs. 8:14-18.

Falla, T. J. & Hancock, R. E. W. (1997) Improved activity of a synthetic indolicidin analog. Antimicrob. Agents Chemother. 41:771-775.

Francisco, J. A. & Georgiou G. (1994) The expression of recombinant proteins on the external surface of Escherichia coli. Biotechnological applications. Ann. N. Y. Acad. Sci. 745:372-382.

Francisco, J. A., Earhart, C. F. & Georgiou, G. (1994) Transport and anchoring of beta-lactamase to the external surface of Escherichia coli. Proc. Natl. Acad. Sci. U.S.A. 89:2713-2717.

Fu, H. & Khosla, C. (1996) Antibiotic activity of polyketide products derived from combinatorial biosynthesis: implications for directed evolution. Mol. Divers. 1:121-124.

Georgiou, G., Stathopoulos, C., Daugherty, P. S., Nayak, A. R., Iverson, B. L. & Curtiss, R. 3^{rd} (1997) Display of heterologous proteins on the surface of microorganisms: from the screening of combinatorial libraries to live recombinant vaccines. Nat. Biotechnol. 15:29-34.

Gilchrist, C. A., Gray, D. A. & Baker, R. T. (1997) A ubiquitin-specific protease that efficiently cleaves the ubiquitin-proline bond. J. Biol. Chem. 272:32280-32285.

Goldman, E. R. & Youvan, D. C. (1992) An algorithmically optimized combinatorial library screened by digital imaging spectroscopy. Biotechnology (NY) 10:1557-1561.

Goldman, E., Fuellen, G. & Youvan, D. C. (1994) Estimation of protein function from combinatorial sequence data using decision algorithms and neural networks. Drug Dev. Res. 33:125-132.

Gottfredsson, M., Erlendsdottir, H., Sigfusson, A. & Gudmundsson, S. (1998) Characteristics and dynamics of bacterial populations during postantibiotic effect determined by flow cytometry. Antimicrob. Agents Chemother. 42:1005-1011.

Hancock, R. E. W. (1997a) Peptide antibiotics. Lancet 349:418-422.

Hancock, R. E. W. (1997b) Antibacterial peptides and the outer membranes of gram-negative bacilli. J. Med. Microbiol. 46:1-3.

Hancock, R. E. W. & Chapple, D. S. (1999) Peptide antibiotics. Antimicrob. Agents Chemother. 43:1317-1323.

Haughland, R. P. (1996) Handbook of Fluorescent Probes and Research Chemicals. Sixth edition. Molecular Probes, Eugene, Oreg., p. 373.

Helmerhorst, E., Van't Hoff, W., Veerman, E., Simoons-Smit, I. & Nieuw Amerongen, A. (1997) Synthetic histatin analogues with broad-spectrum antimicrobial activity. Biochem. J. 326:39-45.

Hershko, A. & Ciechanover, A. (1992) The ubiquitin system for protein degradation. Annu. Rev. Biochem. 61:761-807.

Hill, H. R. & Stockley, P. G. (1996) Phage presentation. Mol. Microbiol. 20:685-692.

Hiramatsu, K. (1998) The emergence of Staphylococcus aureus with reduced susceptibility to vancomycin in Japan. Am. J. Med. 104:7S-10S.

Hoffman, J. A., Kafatos, F. C., Janeway, C. A. & Ezekowitz, R. (1999) Phylogenetic perspectives in innate immunity. Science 284:1313-1318.

Joo, H., Lin, Z. & Arnold, F. H. (1999) Laboratory evolution of peroxide-mediated cytochrome P450 hydroxylation. Nature 399:670-673.

Kang, J. H., Lee, M. K., Kim, K. L. & Hahm, K. -S. (1996) Structure-biological activity relationships of 11-residue highly basic peptide segment of bovine lactoferrin. Int. J. Peptide Protein Res. 48:357-363.

Kelley, K. J. (1996) Using host defenses to fight infectious diseases. Nat. Biotechnol. 14:587-590.

LaBean, T. H., Kauffman, S. A. & Butt, T. R. (1995) Libraries of random-sequence polypeptides produced with high yield as carboxy-terminal fusions with ubiquitin. Mol. Divers. 1:29-38.

LaVallie, E. R. & McCoy, J. M. (1995) Gene fusion expression systems in Escherichia coli. Curr. Opin. Biotechnol. 6:501-506.

LaVallie, E. R., McCoy, J. M., Smith, D. B. & Riggs, P. (1994) Enzymatic and chemical cleavage of fusion proteins. In: Current Protocols in Molecular Biology, Unit 16. John Wiley & Sons, New York.

Leadlay, P. F. (1997) Combinatorial approaches to polyketide biosynthesis. Curr. Opin. Chem. Biol. 1:162-168.

Maloy, W. L. & Kari, U. P. (1995) Structure-activity studies on magainins and other host defense peptides. Biopolymers 37:105-122.

Mata, E. G. (1999) Solid-phase and combinatorial synthesis in beta-lactam chemistry. Curr. Pharm. Des. 5:955-964.

McDaniel, R., Thamchaipenet, A., Gustafsson, C., Fu, H., Betlach, M. & Ashley, G. (1999) Multiple genetic modifications of the erythromycin polyketide synthase to produce a library of novel “unnatural” natural products. Proc. Natl. Acad. Sci. U.S.A. 96:1846-1851.

Merrifield, R. B., Merrifield, E. L., Juvvadi, P., Andreu, D. & Boman, H. G. 1994. In: Antimicrobial Peptides. (Boman, H. G., Marsh, J. & Goode, J. A., eds.) John Wiley & Sons, New York, pp. 5-26.

Moore, J. C. & Arnold, F. H. (1996) Directed evolution of a para-nitrobenzyl esterase for aqueous-organic solvents. Nat. Biotechnol. 14:458-467.

Mortimer, F. C., Mason, D. J. & Gant, V. A. (2000) Flow cytometric monitoring of antibiotic-induced injury in Escherichia coli using cell-impermeant fluorescent probes. Antimicrob. Agents Chemother. 44:676-681.

Nagai, K. & Thogersen, H. C. (1984) Generation of beta-globin by sequence-specific proteolysis of a hybrid protein produced in Escherichia coli. Nature 309: 810-812.

Odell, E., Sarra, R., Foxworthy, M., Chapple, D. & Evans, R. (1996) Antibacterial activity of peptides homologous to a loop region in human lactoferrin. FEBS Lett. 382:175-178.

Oliphant, A. R., Nussbaum, A. L. & Struhl, K. (1986) Cloning of random-sequence oligodeoxynucleotides. Gene 44:177-183.

Piers, K. L., Brown, M. M. & Hancock, R. E. W. (1993) Recombinant DNA procedures for producing small antimicrobial cationic peptides in bacteria. Gene 134:7-13.

Pilon, A. L., Yost, P., Chase, T., Lohnas, G., Burkett, T., Roberts, S. & Bentley, W. (1997) Ubiquitin fusion technology: bioprocessing of peptides. Biotechnol. Prog. 13:374-379.

Pilon, A. L., Yost, P., Chase, T., Lohnas, G. & Bentley, W. (1996) High-level expression and efficient recovery of ubiquitin fusion proteins from Escherichia coli. Biotechnol. Prog. 12:331-337.

Pore, R. S. (1994) Antibiotic susceptibility testing by flow cytometry. Antimicrob. Chemother. 34:613-627.

Porter, J., Deere, D., Pickup, R. & Edwards, C. (1996) Fluorescent probes and flow cytometry: new insights into environmental bacteriology. Cytometry 23:91-96.

Reidhaar-Olson, J. F. & Sauer, R. T. (1988) Combinatorial cassette mutagenesis as a probe of the informational content of protein sequences. Science 241:53-57.

Robey, R. B., Ruiz, O., Santos, A. V., Ma, J., Kear, F., Wang, L. J., Li, C. J., Bernardo, A. A. & Arruda, J. A. (1998) pH-dependent fluorescence of a heterologously expressed Aequorea green fluorescent protein mutant: in situ spectral characteristics and applicability to intracellular pH estimation. Biochemistry 37:9894-9901.

Rodi, D. J. & Makowski, L. (1999) Phage-display technology—finding a needle in a vast molecular haystack. Curr. Opin. Biotechnol. 10:87-93.

Roth, B. L., Poot, M., Yue, S. T. & Millard, P. J. (1997) Bacterial viability and antibiotic susceptibility testing with SYTOX green nucleic acid stain. Appl. Environ. Microbiol. 63:2421-2431.

Sambrook, J., Fritsch, E. F. & Maniatis, T. (1989). Molecular Cloning, A Laboratory Manual. Cold Spring Harbor Laboratory Press, Plainview, NY.

Scott, J. K. & Smith, G. P. (1990) Searching for peptide ligands with an epitope library. Science 249:386-390.

Shafikhani, S., Siegel, R. A., Ferrari, E. & Schellenberger, V. (1997) Generation of large libraries of random mutants in Bacillus subtilis by PCR-based plasmid multimerization. Biotechniques 23:304-310.

Stahl, S. & Uhlen, M. (1997) Bacterial surface display: trends and progress. Trends Biotechnol. 15:185-192.

Steinberg, D. A., Hurst, M. A., Fujii, C. A., Kung, A. H., Ho, J. F., Cheng, F. C., Loury, D. J. & Fiddes, J. C. (1997) Protegrin-1: a broad-spectrum, rapidly microbicidal peptide with in vivo activity. Antimicrob. Agents Chemother. 41:1738-1742.

Stemmer, W. P. C. (1994) DNA shuffling by random fragmentation and reassembly: in vitro recombination for molecular evolution. Proc. Natl. Acad. Sci. U.S.A. 91:10747-10751.

Suller, M. T. & Lloyd, D. (1999) Fluorescence monitoring of antibiotic-induced bacterial damage using flow cytometry. Cytometry 35:235-241.

Swarts, A. J., Hastings, J. W., Roberts, R. F. & von Holy, A. (1998) Flow cytometry demonstrates bacteriocin-induced injury to Listeria monocytogenes. Curr. Microbiol. 36:266-270.

Taguchi, S., Nakagawa, K., Maeno, M. & Momose, H. (1994) In vivo monitoring system for structure-function relationship analysis of the antibacterial peptide apidaecin. Appl. Environ. Microbiol. 60:3566-3572.

Taguchi, S., Ozaki, A., Nakagawa, K. & Momose, H. (1996) Functional mapping of amino acid residues responsible for the antibacterial action of apidaecin. Appl. Environ. Microbiol. 62:4652-4655.

Tong, G. & Nielsen, J. (1996) A convergent solid-phase synthesis of actinomycin analogues—towards implementation of double-combinatorial chemistry. Bioorg. Med. Chem. 4:693-698.

Tsoi, C. J. & Khosla, C. (1995) Combinatorial biosynthesis of ‘unnatural’ natural products: the polyketide example. Chem. Biol. 2:355-362.

Varra, M. & Porro, M. (1996) Group of peptides that act synergistically with hydrophobic antibiotics against gram-negative enteric bacteria. Antimicrob. Agents Chemother. 40:1801-1805.

Wells, J. A. & Lowman, B. (1992) Rapid evolution of peptide and protein binding properties in vitro. Curr. Opin. Biotechnol. 3:355-362.

Westerhoff, H. V., Zasloff, M., Rosner, J. L., Hendler, R. W., De Waal, A., Vaz Gomes, A., Jongsma, A. P. M., Riethorst, A. & Juretic, D. (1995) Functional synergism of the magainins PGLa and magainin-2 in Escherichia coli, tumor cells and liposomes. Eur. J. Biochem. 228:257-264.

Xue, Q., Ashley, G., Hutchinson, C. R. & Santi, D. V. (1999) A multiplasmid approach to preparing large libraries of polyketides. Proc. Natl. Acad. Sci. U.S.A. 96:11740-11745.

Yang, M. M. & Youvan, D. C. (1988) Applications of imaging spectroscopy in molecular biology. I. Screening photosynthetic bacteria. Biotechnology (NY) 6:939-942.

Yang, M. M., Coleman, W. J., Silva, C. M., Dilworth, M. R., Bylina, E. J. & Youvan, D. C. (1997) High resolution imaging microscope (HIRIM). Biotechnology et alia <www.et-al.com>4: 1-16.

Youvan, D. C. (1991) Photosynthetic reaction centers: interfacing molecular genetics and optical spectroscopy. Trends Biochem. Sci. 16:145-149.

Youvan, D. C., Arkin, A. P. & Yang, M. M. (1992) Recursive ensemble mutagenesis. A combinatorial optimization technique for protein engineering. In: Parallel Problem Solving from Nature (Manner, R. & Manderick, B., eds.) Elsevier Science Publishers, pp. 401-410.

Youvan, D. C., Goldman, E., Delagrave, S. & Yang, M. M. (1995) Digital imaging spectroscopy for massively parallel screening of mutants. Methods Enzymol. 246:732-748.

Youvan, D. C. (1994) Imaging sequence space. Nature 369: 79-80.

Youvan, D. C. (1995) Searching sequence space. Biotechnology (NY) 13:722-723.

Youvan, D. C., Silva, C. M., Bylina, E. J., Coleman, W. J., Dilworth, M. R. & Yang, M. M. (1997a) Fluorescence imaging micro-spectrophotometer (FIMS). Biotechnology et alia <www.et-al.com>1:1-16.

Youvan, D. C., Silva, C. M., Bylina, E. J., Coleman, W. J., Dilworth, M. R. & Yang, M. M. (1997b) Calibration of fluorescence resonance energy transfer in microscopy using genetically engineered GFP derivatives on nickel chelating beads. Biotechnology et alia <www.et-al.com>3:1-18.

Zhang, L., Falla, T., Wu, M., Fidai, S., Burian, J., Kay, W. & Hancock, R. E. W. (1998) Determinants of recombinant production of antimicrobial cationic peptides and creation of peptide variants in bacteria. Biochem. Biophys. Res. Commun. 247:674-680.

What is claimed is:

1. A method for determining whether a compound affects cell viability, comprising the steps of:
providing colonies of cells on a support surface, the cells having been transformed with an expression library encoding candidate compounds, wherein expression of the candidate compounds is regulated by an inducible promoter;
exposing the colonies to inducing conditions to induce expression from the inducible promoter;
contacting the colonies of the cells with a viability indicator that produces an optical signal indicative of cell viability; and
determining whether one of the colonies has a desired optical signal, wherein the desired optical signal indicates expression by the colony of a compound that affects cell viability.

2. The method of claim 1, further comprising the step of retrieving DNA from one of the colonies.

3. The method of claim 1, wherein the cells comprise bacteria.

4. The method of claim 1, wherein the indicative signal indicates a non-viable cell.

5. The method of claim 1, wherein the optical signal is a fluorescence signal.

6. The method of claim 1, wherein the optical signal is an absorbance signal.

7. The method of claim 1, wherein the candidate compounds are expressed as fusions with a carrier protein.

8. The method of claim 7, wherein the carrier protein is ubiquitin.

9. The method of claim 7, wherein the candidate compounds are released from the carrier protein prior to the determining step.

10. The method of claim 1, wherein the viability indicator is membrane impermeant.

11. The method of claim 1, wherein the compound disrupts a cell wall or a cell membrane.

12. The method of claim 1, wherein the expression library encodes candidate compounds comprising targeted amino acid sequence mutations.

13. A method for determining whether a compound affects cell viability, comprising the steps of:
providing colonies of cells on a first support surface, the cells having been transformed with an expression library encoding candidate compounds, wherein expression of the candidate compounds is regulated by an inducible promoter;
exposing the colonies to inducing conditions to induce expression from the inducible promoter;
contacting the colonies with a layer of target cells;
contacting the target cells with a viability indicator that produces an optical signal indicative of target cell viability; and
determining whether one of the target cells has a desired optical signal, wherein the desired optical signal indicates expression by the colony adjacent to the target cell of a compound that affects target cell viability.

14. The method of claim 13, further comprising the step of retrieving DNA from one of the colonies.

15. The method of claim 13, wherein the target cells comprise bacteria.

16. The method of claim 13, wherein the indicative signal indicates a non-target viable cell.

17. The method of claim 13, wherein the optical signal is a fluorescence signal.

18. The method of claim 13, wherein the optical signal is an absorbance signal.

19. The method of claim 13, wherein the candidate compounds are expressed as fusions with a carrier protein.

20. The method of claim 19, wherein the carrier protein is ubiquitin.

21. The method of claim 19, wherein the candidate compounds are released from the carrier protein prior to the determining step.

22. The method of claim 13, wherein the viability indicator is membrane impermeant.

23. The method of claim 13, wherein the compound disrupts a target cell wall or a target cell membrane.

24. The method of claim 13, wherein the expression library encodes candidate compounds comprising targeted amino acid sequence mutations.
